(19) United States
(12) Patent Application Publication
(10) Pub. No.: US 2013/0338541 A1
(43) Pub. Date: Dec. 19, 2013

(54) UPPER LIMB BRADYKINESIA AND MOTOR FATIGUE DEVICE

(71) Applicant: Rush University Medical Center, Chicago, IL (US)

(72) Inventor: Leonard Verhagen Metman, Chicago, IL (US)

(21) Appl. No.: 13/918,578

(22) Filed: Jun. 14, 2013

Related U.S. Application Data
(60) Provisional application No. 61/660,468, filed on Jun. 15, 2012.

(51) Int. Cl. A61B 5/11; A61B 5/00

(52) U.S. Cl. CPC A61B 5/1124; A61B 5/4082; A61B 5/4848; A61B 5/7246
USPC 600/595

(57) ABSTRACT

The present disclosure relates to a motor skill assessment device and methods of using the motor skill assessment device. The device includes a gear shaft protruding through a support member. A handle is attached at one end of the gear shaft and a measurement device is located at the other end of the gear shaft. There may be a circuit board in electronic communication with the measurement device and a computer in electronic communication with the circuit board. Methods for quantifying motor skills and methods for evaluating the effect of medical interventions on a patient are also disclosed.

### CROSS-REFERENCE TO RELATED APPLICATIONS

This application is a non-provisional application which claims priority to U.S. Provisional Application Ser. No. 61/660,468 filed Jun. 15, 2012, which is incorporated herein by reference in its entirety.

### BACKGROUND

1. Field of the Invention

The present disclosure generally relates to medical devices and methods of using the same. More particularly, the disclosure relates to medical devices used to monitor the motor skills of a patient and methods for assessing progression of disease, effect of medical interventions, and motor fatigue.

2. Description of the Related Art

Idiopathic Parkinson's disease (PD) is a chronic neurodegenerative disease predominantly affecting elderly people and characterized clinically by symptoms that include bradykinesia (slowed movement), hypokinesia (small amplitude movement), tremor, rigidity, and loss of postural righting reflexes. The onset of movement problems is gradual and asymmetric. Currently, there is no laboratory test for diagnosing or detecting the onset and progression of this nervous system disorder. Therefore, the diagnosis of PD relies on clinical observation of two or three motor signs, such as tremor, rigidity, and/or bradykinesia. By conventional definition, bradykinesia is the essential element without which a diagnosis of PD cannot be made.

Motor signs are traditionally measured with the Unified Parkinson's Disease Rating Scale motor score (UPDRS-III). The UPDRS-III contains tests such as finger tapping, opening and closing of the fist, and pronation/supination of the hand. The rater subjectively assigns a number between 0 (normal) and 4 (severely impaired) to the movement. These tests have limited sensitivity, are not statistically reliable, and comprise a non-continuous scale with limited resolution for detecting small changes in disease progression in early stage PD. Furthermore, patients with cerebellar dysfunction are also bradykinetic but can usually be differentiated from patients with parkinsonism upon clinical examination. Some multisystem disorders, such as multisystem atrophy and the spinocerebellar ataxias, have both parkinsonism and cerebellar dysfunction, and are thus difficult to distinguish.

A computerized software program to assess upper limb motor function has recently been developed and is designated the “BRAIN TEST.” The test is based on the finger tapping test but has the added advantage of providing information on incoordination and dysmetria, clinical signs associated with cerebellar dysfunction. It uses a standard personal computer with the keyboard as the test device. The two targets are the “S” and “;” keys, which are 15 cm apart on the 101/102 keyboard, as well as most notebook size computer keyboards. The target keys are marked with red adhesive paper dots 10 mm in diameter. Test subjects are seated in front of the keyboard at a height that allows their arms to be above the keyboard when their elbows are flexed at 90°. Using the index finger, the subject must alternatively strike the target keys for a period of 60 seconds. Before starting the test, the subjects are told to perform the test as fast and as accurately as possible. Data from the test is analyzed on several variables.

A portable motor system assessment device that allows for rigidity testing in the fingertips has been disclosed in WO/1997/39677. The device includes a rotatable shaft connected to a digital encoder on two sides, thereby allowing the patient to test rotational ability using the fingertips to determine rigidity. The motion utilized in this test is not routine for most patients. Since the rotatable shafts are opposed to one another and not directly comparable, their rotation presents a problem in statistical comparison.

While the foregoing interventions have been helpful, each has a number of drawbacks. The UPDRS-III lacks objectivity, sensitivity, and is subject to variations caused by the person directing the test. The BRAIN TEST resolves the objectivity problem by computerizing the finger tap test but it uses a keyboard that is not universal and comfortable to all potential patients. Thus, there is a need in the art for safe, reliable, and simple methods for monitoring and quantifying bradykinesia in patients with a nervous system disorder.

### BRIEF SUMMARY

In one aspect, the present disclosure relates to a method of quantifying motor skills. The method comprises the step of providing a device including a gear shaft protruding through a support member, a handle disposed at a first end of the gear shaft, and a measurement device disposed at a second end of the gear shaft. The method also comprises the steps of rotating the handle continuously for a period of time, wherein a rotation distance is measured by the measurement device, and transmitting the rotation distance from the measurement device to a computer.

In an additional aspect, the present disclosure relates to a method for evaluating the effect of a medical intervention on a patient. The method comprises the step of providing a device including a gear shaft protruding through a support member, a handle disposed at a first end of the gear shaft, and a measurement device disposed at a second end of the gear shaft. The method also comprises the steps of allowing the patient to rotate the handle continuously for a period of time, wherein a rotation distance is measured by the measurement device, and transmitting the rotation distance from the measurement device to a computer. Next, the method includes the step of performing a medical intervention on the patient. Then, the method includes the steps of allowing the patient to rotate the handle continuously for a second period of time, wherein a second rotation distance is measured by the measurement device, and transmitting the second rotation distance from the measurement device to the computer. Finally, the method includes the steps of comparing the rotation distance achieved before the medical intervention to the second rotation distance and evaluating the effect of the medical intervention.

In another aspect, a motor skill assessment device is provided. The device comprises a gear shaft protruding through a support member, a handle disposed at a first end of the gear shaft, a measurement device disposed at a second end of the gear shaft, a circuit board in electronic communication with the measurement device, and a computer in electronic communication with the circuit board.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE SEVERAL VIEWS OF THE DRAWINGS

A detailed description of the invention is hereafter described with specific reference being made to the drawings in which:

FIG. 1 shows a perspective view of the motor skill assessment device;

FIG. 2 shows a schematic block diagram of the motor skill assessment device;

FIG. 3 shows an exemplary user interface for a computer control of the device;

FIG. 4 shows an exemplary data trace after testing a patient with the motor skill assessment device;

FIG. 5a shows reliability and FIG. 5b shows UPDRS-III comparison test results;

FIG. 6 shows the mean number of turns using the motor skill assessment device over a time period;

FIG. 7 shows the mean amplitude of each turn using the motor skill assessment device over a time period;

FIG. 8 shows an exemplary embodiment of a pacing device;

FIG. 9 shows the mean number of turns using the motor skill assessment device over a time period; and

FIG. 10 shows the mean amplitude of each turn using the motor skill assessment device over a time period.

### DETAILED DESCRIPTION

Various embodiments are described below with reference to the drawings in which like elements generally are referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances, details may have been omitted that are not necessary for an understanding of embodiments disclosed herein, such as conventional fabrication and assembly.

The present disclosure relates to a motor skill assessment device that can be used to evaluate the motor skills of patient. The device includes a handle that, in most aspects, is designed such that it can be comfortably gripped by a patient using the device. The handle can have the shape of a light bulb or a doorknob, for example, and can be rotated/turned in the same manner as one would turn a light bulb or a doorknob. Other shapes for the handle are also possible. The rotation can be clockwise or counterclockwise. As the handle is rotated, the number of complete rotations over a selected period of time can be measured and/or the total rotational distance traveled by the handle can be measured over a selected period of time. While the present disclosure may refer at times to measuring or transmitting a total number of rotations of the handle, it is to be understood that the total rotational distance traveled by the handle, or speed of rotation of the handle, can additionally or alternatively be measured in any of the instances disclosed herein. The total rotational distance traveled, number of rotations, and/or speed of rotation may then be transmitted to a computer.

Digitizing the measured data allows for the presently disclosed methods of analysis to be carried out by a computer. In turn, for example, speed of movement can be measured and the change in speed over time can be monitored objectively for each patient. A declining speed of movement over time can be due to motor fatigue and is a typical feature of PD. Thus, the motor skill assessment device is not only used to evaluate speed but can also be used to monitor motor fatigue in PD.

Additionally, the device can be used to measure changes in amplitude of movement, for example, by holding the speed of rotational movement constant. In certain aspects, patients can turn the handle at a set pace using a pacing device. By way of non-limiting example, the pacing device may be a metronome. The pacing device allows the patient to establish a constant pace of rotation. Since the turning occurs at a set pace, changes in the total number of turns over time will be due to larger or smaller amplitude of the turns. Similarly, changes in a total rotational distance traveled over time will be due to larger or smaller amplitude of the turns. Improvements in speed and amplitude are indicative of improved upper limb mobility in a patient.

The presently disclosed device thus provides a reliable, repeatable, objective, rater-independent measure of bradykinesia over time in patients having nervous system disorders, such as PD. The motor skill assessment device also measures motor control in general, and provides a tool to measure and quantify motor fatigue in patients with PD or other disorders of the central nervous system or musculoskeletal system. In some embodiments, the device can be used in outcome measurements for clinical trials designed to treat these disorders or to monitor individual patient changes in response to treatment. By way of non-limiting example, treatments may include pharmaceutical intervention, surgical intervention, stem cell transplant, nutrient supplements and the like. For example, the motor skill assessment device can be used as a tool that can help in the optimization of deep brain stimulation parameters. Also, the motor skill assessment device can be used as a tool that can help characterize the clinical condition of patients and follow their progression over time thereby monitoring their response to clinical interventions. Moreover, the device can be used as a research tool that can be used to study bradykinesia and fatigue, the response to different interventions, and the response to intra-operative testing.

In one example of using the presently disclosed device, when patients with PD undergo deep brain stimulation (DBS), there will be a time before definitive implantation of the lead that the response to test stimulation needs to be assessed. The method of assessment needs to be accurate, simple, quick, and quantitative in order to make the important decision as to where the DBS lead should be placed. Rather than estimating the improvement in finger tapping as disclosed in the prior art, using the presently disclosed device, which produces a clear cut score (and changes in score), provides an objective basis to make such a decision. Also, after the surgery is completed, patients are followed in the outpatient clinic and the health care provider needs to optimally “set” the parameters of stimulation. An objective, quick measurement of movement is essential in this optimization process, and such a measurement is provided by the presently disclosed device.

In addition to the foregoing uses, the presently disclosed device can also be used to quantify movement in other diseases. For instance, the motor skill assessment device can be employed to follow a multiple sclerosis patient's response to medication, to follow a patient's response to rehabilitation in stroke or other disorders, and/or to monitor patients with myasthenia gravis. Many disorders of the central nervous system (CNS), peripheral nervous system (PNS), and muscular system can be studied with the presently disclosed device in a quantitative manner.

The device includes many different components and all such components may be made from commonly used materials in the art. For example, in some aspects, the device may be made from a plastic so that the motor skill assessment device is MRI compatible and can be used with functional MRI, CT, or other imaging modalities as a research and diagnostic tool. Alternative materials that can be used to manufacture the various components of the presently disclosed device include, but are not limited to, aluminum, plastic, stainless steel, wood, or any combination thereof.

The presently disclosed device provides an easy to operate tool for objective and quantitative speed and amplitude measurements, as well as motor fatigue evaluation, by allowing the patient make a supination and pronation movement in the form of the common, non-skilled task of turning a handle. The device is insensitive to typical PD symptoms, such as tremor and dyskinesia, which can interfere significantly with typical UPDRS-III assessments or peg board type tests. One advantage of using a pronation/supination task to quantify bradykinesia is that such a movement is also used in the gold standard clinical rating scale, the UPDRS-III, but the latter is semi-quantitative as explained in the background section of the present application.

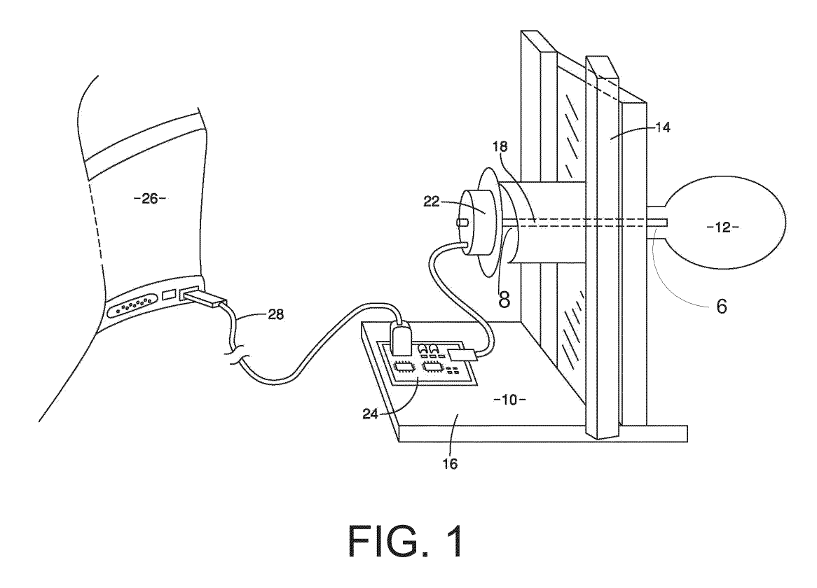

In FIG. 1, a perspective view of an exemplary motor skill assessment device is shown. The device (10) includes various hardware elements. In certain aspects, a handle (12) is attached to a support member (14), such as a vertical stand, and the support member (14) can be mounted to a base (16). In certain aspects, the height of the handle (12) or the position of the handle (12) may be adjustable on the support member (14) to accommodate the particular patient operating the device. As previously mentioned, the handle (12) can have a number of different shapes, such as a bulb/light bulb shape, a doorknob shape, or any other shape that is capable of being manipulated/turned/rotated by a user. In some aspects, the handle (12) may be connected to the support member (14) by a gear shaft (18). The gear shaft has a first end (6) and a second end (8), wherein the handle (12) is connected to the first end (6) and, in certain aspects, a measurement device (22) is connected to the second end (8). The measurement device (22) may be in electronic communication with a circuit board (24) that is in electronic communication with a computer (26). In some aspects (see FIG. 8, for example), the device (10) further includes or is used in connection with a pacing device, such as a metronome (70).

In accordance with certain aspects of the present disclosure, the measurement device (22) is configured to measure, for example, speed, distance traveled, and/or number of handle rotations. In some aspects, the measurement device (22) is a counter or an encoder. An example of an encoder that can be used in connection with the presently disclosed device is the AMT 102V incremental encoder manufactured by CUI, Inc. (Tualiatin, Oreg.). In alternative aspects, a motor with a built-in digital encoder can be used and in further aspects, an optical rotary encoder could be used. As the handle (12) is rotated by the patient, the gear shaft (18) drives the measurement device (22), which can determine the total number of rotations of the handle and/or the total rotational distance traveled by the handle. In certain aspects, this data is then transmitted to a circuit board (24), such as a Phidget high speed encoder or a Phidget high speed encoder interface with USB cabling (Phidgets, Inc. Calgary, Alberta, Canada).

The circuit board (24) communicates the number of rotation data and/or the rotational distance data to a computer (26). The circuit board (24) may communicate the data to the computer (26) in any number of ways, and across any number or combination of wired and/or wireless networks. The circuit board (24) may perform the communication through a wired connection, such as a USB cable (28). The circuit board (24) may additionally or alternatively utilize any number of communication standards, topologies, interfaces, protocols, or methods, to communicate data to the computer (26), including as examples Ethernet, IEEE 802.11a/b/g/n/x/ac/ad, 802.16, Bluetooth, optical, infrared, radiofrequency, universal serial bus, WiFi, WiMAX, Ethernet, cable, satellite, digital subscriber line, Bluetooth, cellular technologies (e.g., 2G, 3G, Universal Mobile Telecommunications System (UMTS), GSM, Long Term Evolution (LTE), or more. In that regard, the circuit board (24) and/or computer (26) may include suitable circuitry and interfaces to communicate data according to any of the communication methods described herein. In certain aspects, the measurement device (22) may additionally or alternatively include communication circuitry and interfaces to communicate with the computer (26) in any of the ways described herein.

In one aspect of using this device, a time period is selected by a test operator using a software program on the computer (26). A patient begins rotating the handle (12) upon receiving a signal provided by the computer (26). Alternatively, the test operator can instruct the patient to begin the test and in other aspects, the test begins as soon as the patient begins to rotate the handle. The measurement device (22) begins recording the number of rotations (or rotational distance traveled) immediately upon the receipt of the start signal from the computer (26). The measurement device (22) stops recording the number of rotations (or rotational distance traveled) upon receipt of a stop signal from the computer (26) based on the testing time period selected by the operator.

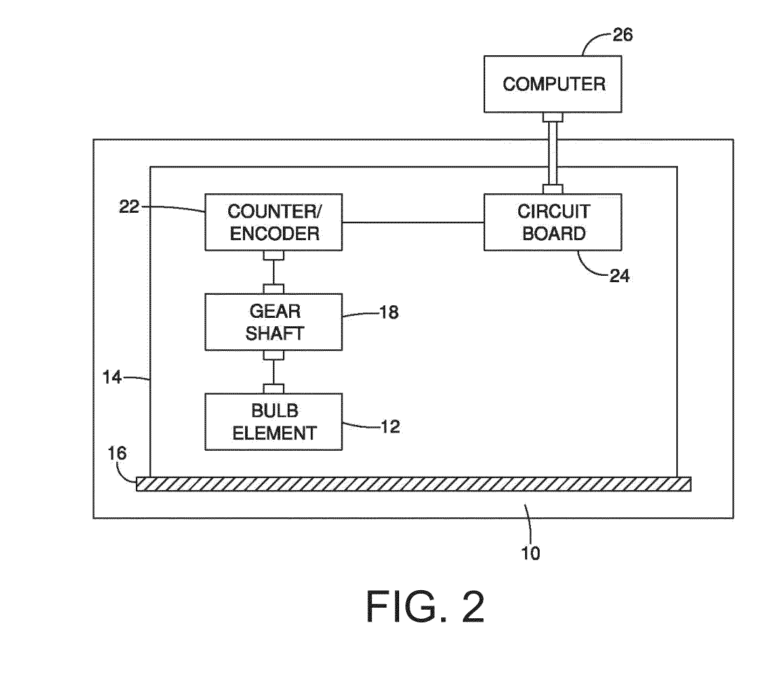

In FIG. 2, a schematic block diagram of an aspect of the device is shown. The device (10) is mounted on a support member (14) connected to a base (16). The handle (12) is connected to the gear shaft (18) whereby rotation of the handle (12) causes rotation of the gear shaft (18). Rotation of the gear shaft (18) is measured by the measurement device (22) and the number of rotations and/or the rotational distance traveled may be transmitted (wirelessly or through a wire) to the circuit board (24). In turn, the circuit board (24) transmits this information to the computer (26) either wirelessly or through a wire, such as a USB cable and/or any of the communication techniques described herein.

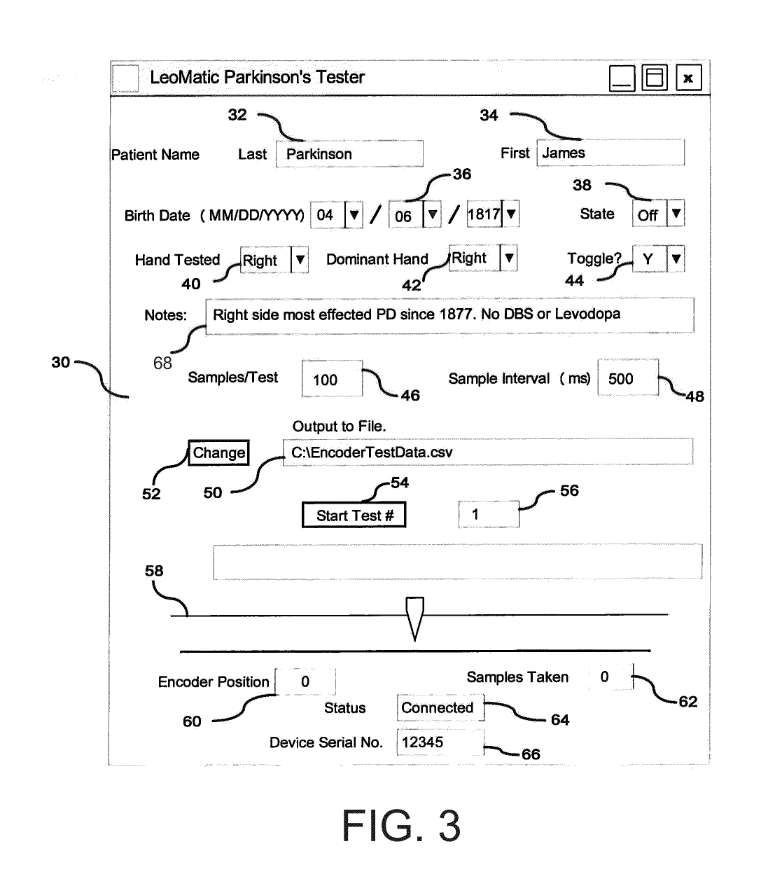

FIG. 3 shows a controller interface contained in the computer software program used in connection with the presently disclosed motor skill assessment device. It is to be understood that FIG. 3 merely depicts one potential aspect of a controller interface, other aspects can include fields not shown in FIG. 3, and still other aspects may not include certain fields shown in FIG. 3.

The computer can be a laptop, desktop, notebook, smart phone, or any other computing device. The computer can include user interfaces, such as one or more input devices (e.g., a mouse, a keyboard, etc.) and one or more graphical user interfaces (e.g., a display, monitor, or other visual interface). The computer will display a screen (30) with various input fields. These input fields can have pre-loaded information such as “on/off' or “yes/no” or the test operator can input information into the fields using a keyboard or other appropriate input device. Particular input fields can be selected according to the needs of the particular test being conducted.

In certain aspects, input fields can include a “last name” field (32), a “first name” field (34), and a “birth date” field (36), to provide personal information about the test patient that allows the patient to be tracked properly. Other patient identifiers can also be included for patient tracking purposes. A “state” field (38) provides information about the condition of the patient. This field can be used in PD patients who are determined to be in the well-medicated state or in the un-medicated state, for example.

The test operator will determine which hand will be tested and input this information into a “hand tested” field (40). The operator will also determine the patient's dominant hand and place this information in a “dominant hand” field (42). A “toggle” field (44) provides the opportunity to switch between a patient's right and left hands automatically and a “notes” field (68) allows the operator to input information about the test patient that may be relevant to the diagnosis or condition. The “samples/test” field (46) denotes the number of samples collected and the “sample interval” field (48) gives the test controller the ability to determine the sample interval (shown in FIG. 3 in milliseconds) so that the frequency of sampling can be varied as desired. The test operator determines where the data should be saved once generated and names the appropriate file in the “output to file” field (50). In certain aspects, the file name is standardized but the test operator has the option of changing the file name by selecting the “change” button (52) associated with the “output to file” field (50).

The test is started by pressing the “Start Test #” button (54) and the test operator can input the test number in the test number field (56). Alternatively, the test number field (56) can be populated by the software program. A test progress bar (58) is shown that enables the test operator to monitor the progress of the test. An “encoder position” field (60) provides information about the position of the encoder (or other type of measurement device) and thus indicates the cumulative turns of the handle. Alternatively, the “encoder position” field (60), or a similar field, can provide a rotational distance traveled by the handle measurement. For example, if a full rotation of the handle would be equivalent to a circumference or rotational distance of about 6 inches but the patient only rotated the handle one-half of a rotation, a measurement of about 3 inches would populate in the field.

A “samples taken” field (62) enables the program to determine the number of samples generated by a test patient and can automatically be filled in by the program. The device status is shown in a “status” field (64) to enable the test controller to assure that the device is working properly. For validation and comparison purposes, a “tracking” field (66) can be included, which includes the serial number of the device.

The software programs disclosed herein enable a clinical user to obtain quantitative, objective measurements of the degree of Parkinson's symptoms in patients and the subsequent responses of such patients to treatment therapies. In certain aspects, the software is written in visual basic, although any compatible programming code can be used, such as Java, C, C++, assembly, javascript, python, and more. In some aspects, the software programs disclosed herein may be implemented as a distributed application, and a server may implement various portions or functionality of the software programs and the computer (26) may act as client. The software programs disclosed herein may alternatively be implemented as hardware, firmware, or combinations thereof, e.g., by the computer (26).

For a given test, a specified number of samples are taken at the desired frequency, and the measurement device values/measurements are recorded at the conclusion of the test to, for example, an Excel-compatible “.csv” file. The clinician is then able to apply existing Excel analysis and graphing tools to meaningfully display the results. A column-heading row can be automatically written to any new output .csv file, with the headings directly corresponding to screen fields. In some aspects, the measurement device values/measurements may be stored in a system database or other storage devices internal or external to the computer (26). In that regard, the system database or other databases may catalog and track the measurement device values/measurements for one or more patients.

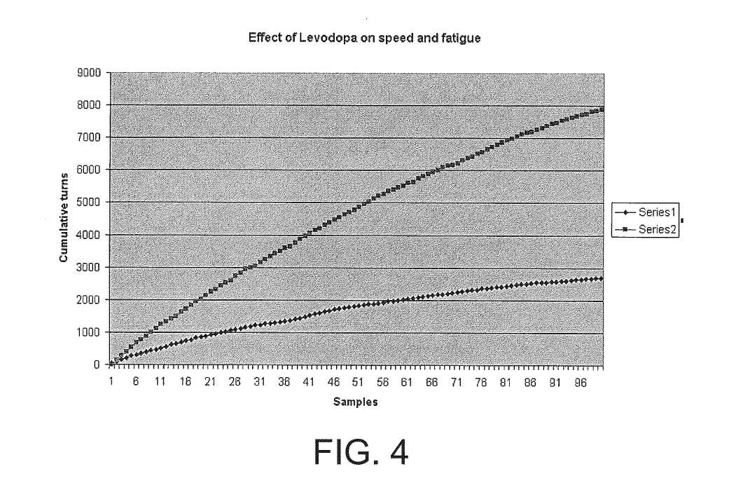

FIG. 4 shows an exemplary trace of data generated from a patient tested with the presently disclosed device. This type of trace appears on the computer screen after the data has been processed by the program. In certain aspects, the data are automatically written to an Excel spreadsheet from which graphs can be generated to visualize the data. The vertical axis in FIG. 4 shows the number of turns. Due to the specific measurement device used to generate this data, each full turn scores 190 on the measurement device, although this score can vary by the type of measurement device. Therefore, the numbers on the vertical axis are divided by 190 to get the actual number of full rotations. The number of samples over time are plotted on the horizontal axis.

A trial of 50 seconds at a sampling rate of 500 milliseconds will show numbers from 0 to 100. The graph can be used to depict two types of results. First, the graph shows the absolute number of turns at the end of the trial period. This is a measure of cumulative distance of handle rotations and therefore, an indication of the patient's motor performance as far as speed and amplitude of movement are concerned. Second, the output visualizes fatigue. If there were no fatigue during the trial, the cumulative handle turns would be depicted as a straight line on the graph. However, when motor fatigue is present, the graph starts to bend as each subsequent turn is characterized by reducing speed and amplitude of the rotation. Analyses can be done to determine the change of rotations per sample or per several samples as desired, and fatigue can thus be quantified using various statistical analyses.

For example, a continuous difference in measurements at various time intervals during the test (including, but not limited to, 5 second intervals, 10 second intervals, 15 second intervals, 20 second intervals, 30 second intervals, or any other selected time interval) can be analyzed using any valid statistical method (such as T-test, ANOVA). A decrease in the number of rotations over time is indicative of fatigue and changes of this measurement over the life of the patient correlates with the progression of the disease. Improvements in the number of rotations over time and over the patient's evaluative period are indicative of the efficacy of the selected intervention.

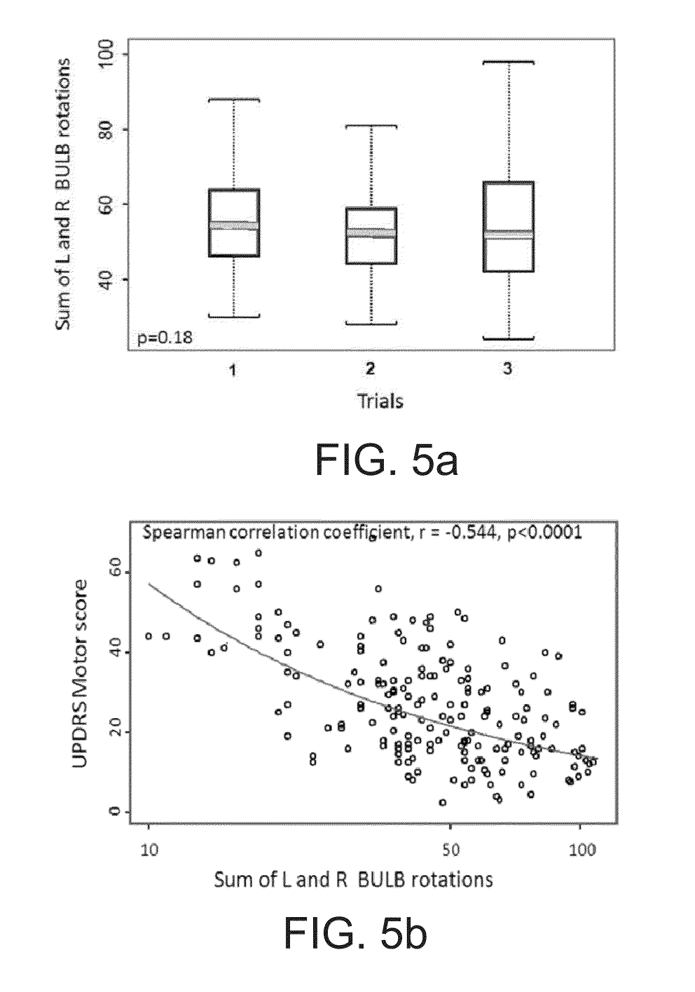

FIG. 5 shows results from a test of the reliability and correlation of the device when compared with the UPDRS-III test. The test-retest reliability is shown in FIG. 5a wherein a normal patient was tested three times for total left and right rotations with a rest period in between. No statistical difference was observed between the trials. Correlation with the UPDRS-III is shown in FIG. 5b. As can be seen, the device demonstrates excellent validity and test-re-test reliability.

In view of the foregoing disclosure, it can be seen that the present application provides a motor skill assessment device having a plurality of uses and that is capable of revealing or evaluating certain characteristics of a patient. In connection with the presently disclosed device, in one aspect of the present disclosure, a method of quantifying motor skills is provided. While using any aspect of the presently disclosed motor skill assessment device, a patient would rotate the handle continuously for a period of time and the rotation distance or number of rotations would be measured by the measurement device. In some embodiments, the measurement device would transmit the rotation distance or number of rotation data to a computer.

In connection with the method disclosed in the preceding paragraph, the method may include a second test conducted at a later time period, such as the next day, the next week, the next month, etc., whereby the patient would once again rotate the handle continuously for a second period of time and a second rotation distance or number of rotations would be measured by the measurement device. The measurement device would then transmit the second rotation distance or number of rotations to the computer. At that time, one could compare, for example, the rotation distance or number of rotation data obtained from the test carried out in a first test to the rotation distance or number of rotation data obtained from the test carried out in the second test.

The comparison step can be executed by a computer. The time period selected to conduct these tests can be selected based on the evaluation to be completed. In certain aspects, the time period is selected from the group consisting of about 10 seconds, about 15 seconds, about 20 seconds, about 25 seconds, about 30 seconds, about 35 seconds, about 40 seconds, about 45 seconds, about 50 seconds, about 55 seconds, about 60 seconds, and greater than 60 seconds.

The methods disclosed herein may include a rotation cycle or multiple rotation cycles. In some aspects, a rotation distance (or number) from a beginning period of the rotation cycle is compared to a rotation distance from an ending period of the rotation cycle. More rotations recorded in the beginning period than at the ending period would be indicative of motor fatigue. In some aspects, the handle is rotated at a set pace during the rotation cycle, thereby substantially keeping a uniform speed of rotation throughout the rotation cycle. A change in the total distance of rotation (or number of rotations) between the beginning period and the ending period can be compared. Since speed is being held constant, any change in the distance of rotation or number of rotations would be due to changes in amplitude of rotation. A rotation cycle can be for any period of time. In certain aspects, the rotation cycle is about 60 seconds, the beginning period is the first 10 seconds of the rotation cycle, and the ending period is the last 10 seconds of the rotation cycle.

The present disclosure also provides methods for evaluating the effect of a medical intervention on a patient. In one aspect, the presently disclosed motor skill assessment device is provided and a patient rotates the handle continuously for a period of time. The rotation distance (or number of rotations) is measured by the measurement device. The measurement device then transmits the rotation distance to a computer and the information is stored. Then, a medical intervention is performed on the patient. After the medical intervention (or plurality of medical interventions), the patient once again rotates the handle continuously for a second period of time, wherein a second rotation distance (or number of rotations) is measured by the measurement device. The measurement device then transmits rotation distance to the computer and the rotation distance achieved before the medical intervention is compared to the rotation distance achieved after the medical intervention. Based on these numbers, the effect of the medical intervention can be evaluated. In certain aspects, the medical intervention is selected from the group consisting of a pharmaceutical treatment, a surgical procedure, physical therapy, and any combination thereof. Such a method can also be used to track the progression of a particular disease or medical condition of a patient.

### EXAMPLES

In a clinical example, seventy-seven PD patients were recruited from the Rush University Medical Center Movement Disorder clinic. Patients with parkinsonism caused by something other than PD and patients with the inability to perform the task due to co-morbidities (cognitive limitations and/or physical limitations) were excluded. The patients were examined on their regular medication regimen. Twenty-four healthy controls, matched for age and gender, were also studied.

First, subjects were asked to turn the handle of the presently disclosed device as fast as possible for 50 seconds. To determine speed, the number of clockwise turns was measured and divided by time. To measure fatigue, the speed in the first 5 seconds was compared to the speed in the last 5 seconds of handle turning. Second, patients were asked to turn the handle as far as possible to the rhythm of a metronome with a frequency of 1 Hz, to measure the amplitude of their movement. By setting the pace (at a slow enough rate that all patients could manage) speed was controlled and thus, any change in number of turns over time would have to be due to changes in amplitude rather than speed. To measure fatigue, the amplitude in the first and last 5 seconds of handle turning was compared.

In PD patients, the side more affected was compared to the side less affected. In controls, the dominant hand was compared to the non-dominant hand. In PD patients, dominance was corrected by normalizing data to the non-dominant hand. Paired t-tests and Wilcoxon signed rank tests were used for statistical comparisons.

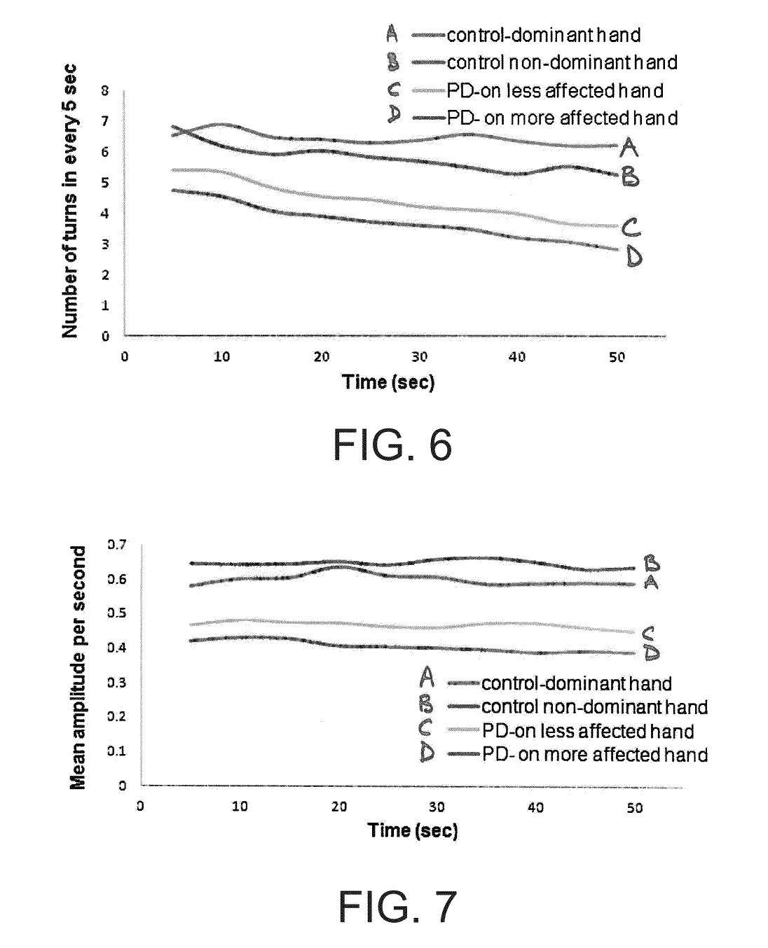

PD patients showed a lower number of turns than healthy controls, as would be expected. There was a significant difference in number of turns between the first 5 seconds and last 5 seconds. PD patients in both the “on” and “off' states showed fatigue in both the more affected hand and the less affected hand. Healthy controls showed fatigue only in the non-dominant hand. These results are depicted in Table 1 and FIG. 6.

TABLE 1

| | | | No Turns | | | |
|---|---|---|---|---|---|---|
| | | | in | No. Turns in | | |
| | | Total Turns | 1-5 sec | 46-50 sec | | |
| | N | (SD) | (SD) | (SD) | % Change | P-value |
| PD Patients - More | 77 | 37.26 | 4.74 | 2.84 | −34.1 | <0.0001 |
| affected hand | | (19.09) | (2.37) | (1.78) | (34.9) | |
| PD Patients - less | 76 | 44.03 | 5.41 | 3.58 | −24.8 | <0.0001 |
| affected hand | | (19.09) | (3.16) | (1.87) | (39.9) | |
| Healthy Controls - | 24 | 64.24 | 6.53 | 6.21 | −0.16 | 0.66 |
| Dominant Hand | | (24.31) | (2.86) | (1.97) | (22) | |
| Healthy Controls - | 25 | 57.88 | 6.83 | 5.22 | −14.3 | 0.003 |
| non-dominant hand | | (18.64) | (3.09) | (1.72) | (34.1) | |

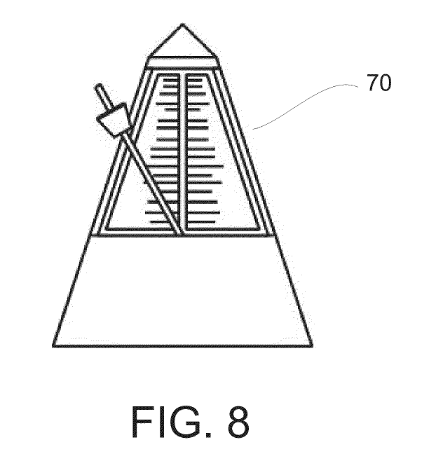

When examining the amplitude of rotations, PD patients showed a lower mean amplitude of turns than healthy controls, as anticipated. There was a significant decrease in amplitude (fatigue) from the first 5 seconds to the last 5 seconds in the more affected hand of PD patients. There was no fatigue of amplitude in the less affected hand of PD patients or in the controls. This data can be seen in Table 2 and FIG. 7.

TABLE 2

| | | Mean | | | | |
|---|---|---|---|---|---|---|
| | | Amplitude | Mean Size of 1 | Mean size of 1 | | |
| | | over | turn in 1-5 sec | turn in 46-50 | % change | |
| | N | 50 sec (SD) | (SD) | Sec (SD) | (SD) | P-value |
| PD Patients - More | 51 | 0.41 | 0.42 | 0.39 | −6.7 | 0.03 |
| affected hand | | (0.17) | (0.17) | (0.18) | (28.8) | |
| PD Patients - less | 51 | 0.47 | 0.47 | 0.45 | −5 | 0.20 |
| affected hand | | (0.22) | (0.19) | (0.22) | (24.8) | |
| Healthy Controls - | 19 | 0.60 | 0.58 | 0.59 | 5.6 | 0.44 |
| Dominant Hand | | (0.14) | (0.19) | (0.12) | (17.3) | |
| Healthy Controls - | 19 | 0.65 | 0.64 | 0.63 | −.06 | 0.66 |
| non-dominant hand | | (0.14) | (0.16) | (0.15) | (12.8) | |

Levodopa (LD) is commonly used in connection with the treatment of PD. It is known that LD improves the speed of movement but there is debate if it also improves the amplitude. The objective of this next study was to use the motor skill assessment device to evaluate the effects of LD on speed and amplitude.

PD patients undergoing OFF-ON testing as part of a DBS evaluation participated in the study. Those with less than 25% difference between OFF and ON in UPDRS-III items 23-25 were excluded. They were tested in the defined OFF state, 12 hours after their last dose of PD medication, and again after a supra-normal dose of LD.

First, subjects were asked to turn the handle of the motor skill assessment device as fast as possible for 50 seconds. The number of clockwise turns was measured to determine speed. The number of turns during five-second bins at the beginning (‘second 6-10’) and at the end (‘second 46-50’) of handle turning was compared to measure fatigue.

Second, subjects were asked to turn the handle as far as possible to the rhythm of a metronome with a frequency of 1 Hz to measure the amplitude of their movement. By setting the pace (at a slow enough rate that all patients could manage) speed was controlled and therefore, any change in number of turns over time would have to be due to changes in amplitude rather than speed. The amplitude in the first (‘second 1-5’) and last (‘second 46-50’) five seconds of handle turning was compared to measure fatigue. Paired t-tests, Wilcoxon signed rank tests and Spearman correlations were used for statistical comparisons.

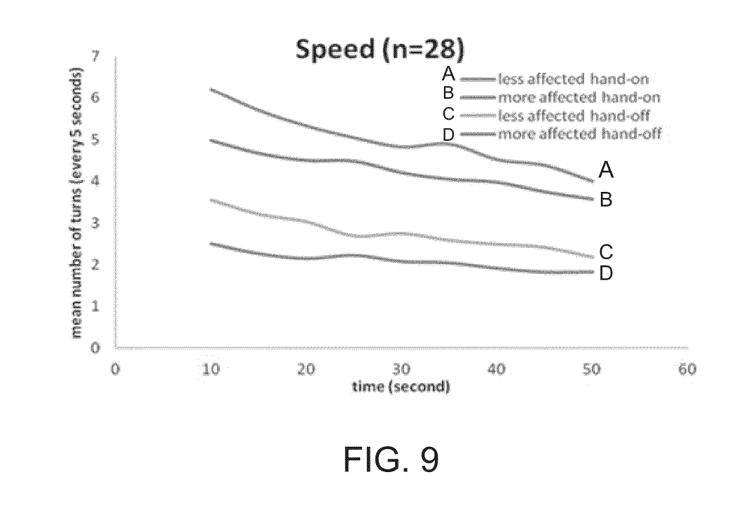

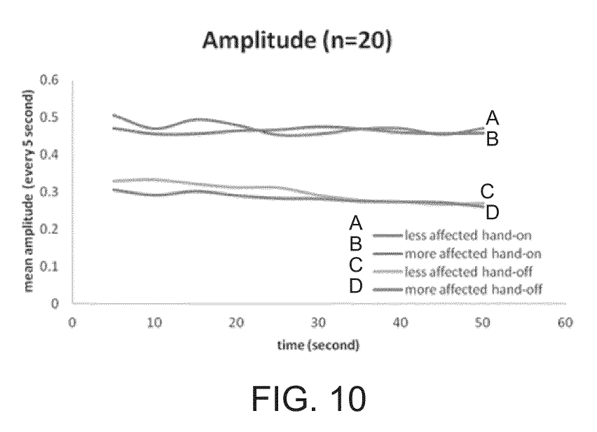

The results of these studies are summarized in Tables 3-4 and FIGS. 9-10.

With respect to speed, PD patients (N=28) in the ON state showed higher scores than in the OFF state as anticipated. There was a significant difference in number of turns during ‘second 5-10’ vs ‘second 45-50,’ indicating fatigue in both the OFF and ON states. Speed decreased faster in the ON than the OFF state. As can be seen in FIG. 9, all curves show a downward slope, indicating fatigue of speed.

TABLE 3
Speed evaluation

| | | | Mean Difference |
|---|---|---|---|
| More affected | | | between |
| hand | ON | OFF | ON and OFF |
| Total # turns | 42.7 ± 18.1 | 21.1 ± 14.6 | 21.3 ± 14.1 |
| in 50 secs | | | P < 0.0001 |
| # turns during | 5.0 ± 1.9 | 2.5 ± 1.4 | |
| ‘sec 6-10’ | | | |
| # turns during | 3.6 ± 2.1 | 1.8 ± 1.7 | |
| ‘sec 46-50’ | | | |
| Change in # | −1.4 ± 1.7 | −0.7 ± 0.9 | −0.7 ± 1.4 |
| turns during | P = 0.0001 | P = 0.0005 | P = 0.01 |
| ‘sec 6-10’ vs | | | |
| ‘sec 46-50’ | | | |

With respect to amplitude, PD patients (N=20) showed a higher mean amplitude of turns in the ON state than in the OFF state, confirming that LD improves the amplitude of movement. There was a significant decrease in amplitude (fatigue) from ‘second 1-5’ to ‘second 45-50’ in the OFF state only. As can be seen in FIG. 10, fatigue was present in the OFF state but not in the ON state.

TABLE 4
Amplitude evaluation

| | | | Mean Difference |
|---|---|---|---|
| More affected | | | between |
| hand | ON | OFF | ON and OFF |
| Mean amplitude | 0.46 ± 0.19 | 0.28 ± 0.19 | 0.18 ± 0.22 |
| over 50 secs | | | P = 0.001 |
| Mean amplitude | 0.47 ± 0.18 | 0.31 ± 0.18 | |
| during | | | |
| ‘second 1-5’ | | | |
| Mean amplitude | 0.46 ± 0.21 | 0.26 ± 0.2 | |
| during | | | |
| ‘second 46-50’ | | | |
| Change in mean | −0.01 ± 0.12 | −0.05 ± 0.08 | 0.03 ± 0.13 |
| amplitude from | NS | P = 0.01 | P = 0.29 |
| ‘second 1-5’ to | | | |
| ‘second 46-50’ | | | |

These examples show that the presently disclosed motor skill assessment device is a useful tool to separately evaluate both speed and amplitude of hand movements in PD. The device thus allows quantification of motor fatigue of hand movements in PD. The results of the experimental studies show that the device is useful to assess the effect of interventions (medical, surgical, etc.) on bradykinesia and fatigue, and that the device can be useful in clinical trials and clinical practice for longitudinal follow-up of PD patients.

Although the present disclosure describes different methods that can be used in connection with the motor fatigue testing device for patients with movement disorders, it is contemplated that the device can also be used for patients with other neurological disorders, including stroke, multiple sclerosis, traumatic brain injury, neuromuscular disorders, and the like. Furthermore, the device can also be used during various imaging modalities. Thus, the present disclosure should not be read to limit the use of the device to movement disorders.

All of the devices, components, and methods disclosed and claimed herein can be made and executed without undue experimentation in light of the present disclosure. In addition, unless expressly stated to the contrary, use of the term “a” is intended to include “at least one” or “one or more.” For example, “a device” is intended to include “at least one device” or “one or more devices.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

The systems and devices described above, such as the measurement device (22), circuit board (24), and computer (26), can be implemented in many different ways in many different combinations of hardware, software, or both hardware and software. For example, all or parts of the system may include circuitry in a controller, a microprocessor, or an application specific integrated circuit (ASIC), or may be implemented with discrete logic or components, or a combination of other types of analog or digital circuitry, combined on a single integrated circuit or distributed among multiple integrated circuits. All or part of the logic described above may be implemented as instructions for execution by a processor, controller, or other processing device and may be stored in a tangible or non-transitory machine-readable or computer-readable medium such as flash memory, random access memory (RAM) or read only memory (ROM), erasable programmable read only memory (EPROM) or other machine-readable medium such as a compact disc read only memory (CDROM), or magnetic or optical disk. Thus, a product, such as a computer program product, may include a storage medium and computer readable instructions stored on the medium, which when executed in an endpoint, computer system, or other device, cause the device to perform operations according to any of the description above.

The processing capability of any disclosed systems and devices may be distributed among multiple system components, such as among multiple processors and memories, optionally including multiple distributed processing systems. Parameters, databases, and other data structures may be separately stored and managed, may be incorporated into a single memory or database, may be logically and physically organized in many different ways, and may implemented in many ways, including data structures such as linked lists, hash tables, or implicit storage mechanisms. Programs may be parts (e.g., subroutines) of a single program, separate programs, distributed across several memories and processors, or implemented in many different ways, such as in a library, such as a shared library (e.g., a dynamic link library (DLL)). The DLL, for example, may store code that performs any of the system processing described above.

What is claimed is:

1. A method of quantifying motor skills comprising:
a) providing a device comprising a gear shaft protruding through a support member, a handle disposed at a first end of the gear shaft, and a measurement device disposed at a second end of the gear shaft,
b) rotating the handle continuously for a period of time, wherein a rotation distance is measured by the measurement device, and
c) transmitting the rotation distance from the measurement device to a computer.

2. The method of claim 1, further comprising the steps of:
d) rotating the handle continuously for a second period of time, wherein a second rotation distance is measured by the measurement device,
e) transmitting the second rotation distance from the measurement device to the computer, and
f) comparing the rotation distance from step b) to the second rotation distance from step d).

3. The method of claim 2, wherein the comparing step is performed by the computer.

4. The method of claim 2, wherein the period of time from step b) and the second period of time from step d) are selected from the group consisting of about 10 seconds, about 15 seconds, about 20 seconds, about 25 seconds, about 30 seconds, about 35 seconds, about 40 seconds, about 45 seconds, about 50 seconds, about 55 seconds, about 60 seconds, and greater than 60 seconds.

5. The method of claim 2, wherein the period of time from step b) and the second period of time from step d) are the same.

6. The method of claim 1, further comprising a rotation cycle, wherein a rotation distance from a beginning period of the rotation cycle is compared to a rotation distance from an ending period of the rotation cycle.

7. The method of claim 6, wherein the handle is rotated at a set pace during the rotation cycle, thereby substantially keeping a uniform speed of rotation throughout the rotation cycle, and measuring a change in a total distance of rotation between the beginning period and the ending period.

8. The method of claim 6, wherein the rotation cycle is about 60 seconds, the beginning period is about a first 10 seconds of the rotation cycle, and the ending period is about a last 10 seconds of the rotation cycle.

9. The method of claim 1, wherein the measurement device transmits the rotation distance to a circuit board and the circuit board transmits the rotation distance to the computer, optionally wherein the rotation distance is transmitted wirelessly.

10. A method for evaluating the effect of a medical intervention on a patient comprising:
a) providing a device comprising a gear shaft protruding through a support member, a handle disposed at a first end of the gear shaft, and a measurement device disposed at a second end of the gear shaft,
b) allowing the patient to rotate the handle continuously for a period of time, wherein a rotation distance is measured by the measurement device,
c) transmitting the rotation distance from the measurement device to a computer,
d) performing a medical intervention on the patient,
e) allowing the patient to rotate the handle continuously for a second period of time, wherein a second rotation distance is measured by the measurement device,
f) transmitting the second rotation distance from the measurement device to the computer,
g) comparing the rotation distance from step b) to the second rotation distance from step e), and
h) evaluating the effect of the medical intervention.

11. The method of claim 10, wherein the medical intervention is selected from the group consisting of a pharmaceutical treatment, a surgical procedure, physical therapy, and any combination thereof.

12. The method of claim 10, wherein the period of time from step b) and the second period of time from step e) are the same.

13. The method of claim 10, wherein step b) comprises a first rotation cycle and step e) comprises a second rotation cycle, wherein a rotation distance from a beginning period of the first rotation cycle is compared to a rotation distance from a beginning period of the second rotation cycle

14. The method of claim 13, wherein a rotation distance from an ending period of the first rotation cycle is compared to a rotation distance from an ending period of the second rotation cycle.

15. The method of claim 13, wherein the handle is rotated at a set pace during the first and second rotation cycles, thereby substantially keeping a uniform speed of rotation throughout the first and second rotation cycles, and measuring a change in a total distance of rotation between the first rotation cycle and the second rotation cycle.

16. The method of claim 14, wherein the first and second rotation cycles are about 60 seconds, the beginning period of the first and second rotation cycles is about a first 10 seconds of the rotation cycles, and the ending period of the first and second rotation cycles is about a last 10 seconds of the rotation cycles.

17. A motor skill assessment device comprising:
a gear shaft protruding through a support member,
a handle disposed at a first end of the gear shaft,
a measurement device disposed at a second end of the gear shaft, and
a computer in electronic communication with the measurement device.

18. The device of claim 17, wherein the measurement device is in electronic communication with a circuit board and the circuit board is in electronic communication with the computer.

19. The device of claim 17, wherein the computer comprises a software program for monitoring and evaluating motor fatigue.

20. The device of claim 17, further comprising a pacing device.
